(19) United States
(12) Patent Application Publication
(10) Pub. No.: US 2009/0061460 A1
(43) Pub. Date: Mar. 5, 2009

(54) TEST APPARATUS, PRODUCTION METHOD THEREFOR AND TEST METHOD

(75) Inventors: Eloisa Lopez-Calle, Ludwigshafen (DE); Juergen Oberstrass, Habichtswald (DE); Werner Siekmann, Lichtenfels (DE)

Correspondence Address: PATENT CENTRAL LLC;Stephan A. Pendorf, 1401 Hollywood Boulevard, Hollywood, FL, 33020, US

(73) Assignee: ANALYTICON BIOTECHNOLOGIES AG, Lichtenfels (DE)

(21) Appl. No.: 12/200,335

(22) Filed: Aug. 28, 2008

Related U.S. Application Data
(63) Continuation of application No. PCT/EP2007/001672, filed on Feb. 27, 2007.

(30) Foreign Application Priority Data
Feb. 28, 2006 (DE) 10 2006 009 516.2

(51) Int. Cl. G01N 33/53; C12M 1/00

(52) U.S. Cl. 435/7.9; 435/288.7

(57) ABSTRACT

Test apparatus for detecting an analyte (A) contained in a sample liquid, comprising a dry porous carrier (10), on which a selective binder (12), capable of selectively binding the analyte (A) after the carrier (10) is wetted with the sample fluid, is arranged in a reaction zone (20), wherein furthermore arranged in the reaction zone (20) are: a complex, formed by a biomarker (B) and the first complexed reporter partner of a pair of reporters, interacting to produce an optically detectable signal, of reporter enzyme (E) and reporter substrate (S), and the second, free reporter partner of the pair of reporters, wherein the biomarker (B), in terms of the selectivity of the binding capacity of the selective binder (12), is equivalent to the analyte (A) and competitive therewith and wherein binding of the selective binder (12) to the complexed biomarker (B) impedes the interaction between the complexed and the free reporter partners.

### BACKGROUND OF THE INVENTION

1. Field of the Invention

The invention relates to a test apparatus for detecting an analyte contained in a sample liquid, comprising a dry porous carrier on which a selective binder is arranged in a reaction zone, said binder being capable of selectively binding the analyte once the carrier has been wetted with the sample fluid.

The invention further relates to a method for producing a test apparatus as well as a method for detecting an analyte in a liquid sample.

2. Related Art of the Invention

A generic test apparatus is generally known under the name “lateral-flow” test and is described in particular in EP 0 291 194 B2. The lateral flow test involves a dry, porous carrier on which a specific binding reagent, e.g. a specific monoclonal antibody against the analyte, is dried on in a so-called start zone. The antibody is tagged with a tagging particle, e.g. a gold or latex particle. It is dried on the carrier in such a way that, when the carrier is in a dry state, it is fixed to the surface of the latter, and when the carrier is in a wetted state, it can move freely therein. In order to use the test, a sample that is presumed to contain the analyte is applied in the start zone. The analyte contained in the sample liquid binds with the tagged antibody and is transported together with the latter, by capillary forces, from the start zone and along the longitudinal extent of the carrier. A binding reagent that is also specific for the analyte is durably immobilized in a detection zone located downstream of the start zone. This is, for example, another antibody that is specifically directed against a different epitope of the analyte than the tagged antibody. The detection antibody is bound to the surface of the carrier in such a way that it also remains fixed in the detection zone when the carrier is in a wetted state. If the analyte coupled to the tagged antibody enters into the detection zone, it binds in a so-called sandwich reaction with the immobilized antibody and this leads to a durable colouring of the detection zone due to the particle tagging. If the sample fluid contains no analyte, no binding occurs between the two antibodies involved, so that the tagged antibody passes through the detection zone and no colouring of the detection zone occurs.

The test can be used for a plurality of analytes. One particularly important use is as a pregnancy test to detect hCG.

Its low sensitivity towards low analyte concentrations is a disadvantage of this known test apparatus. In fact, each molecule of analyte contributes to the colouring of the detection zone, only by binding exactly one tagging particle. The known test apparatus is therefore unsuitable for tests in which analytes that are present only in low concentrations must be detected.

U.S. Pat. No. 3,817,837 describes an enzymatically amplified detection method for an analyte in a sample liquid. To start with, this method comprises the steps of providing the following reaction elements:
a selective binder that is capable of selectively binding the analyte,
a complex formed from a biomarker and the first, complexed reporter partner of a pair of reporters, consisting of a reporter enzyme and a reporter substrate, that interact to generate an optically detectable signal. The biomarker is a substance that is equivalent to the analyte with regard to the selectivity of the binding capability of the selective binder. This includes the possibility of using analyte molecules themselves as biomarkers,
the second, free reporter partner of the pair of reporters.

A pairing of an enzyme and the associated substrate is referred to as a reporter pair in which an enzymatic conversion of the reporter substrate by the reporter enzyme gives rise to an optically detectable signal, e.g. a coloration. The complex consisting of a biomarker and a first reporter partner is designed in such a way that a binding of the selective binder with the biomarker hinders the enzymatic conversion of the reporter substrate by the complexed reporter enzyme, i.e. it reduces or completely eliminates the efficiency of the conversion. In the known method, all the reaction elements are pipetted together with the sample liquid to be analyzed, in a predetermined sequence and in predetermined amounts, into a reaction vessel. If the sample liquid contains an analyte, the analyte molecules compete with the biomarker molecules for binding sites of the selective binder. The more analyte that is contained in the sample liquid, the smaller the number of biomarker/reporter partner complexes that are coupled with a selective binder molecule; and the less the interaction between the reporter enzyme and the reporter substrate is impaired, which leads to a correspondingly stronger, optically detectable signal.

This known method is very sensitive because, on the one hand, it uses a competitive approach, and on the other hand it exhibits an amplification effect because each analyte molecule contributes to a plurality of substrate conversions; however, its use is complicated and depends on the correct observance of the predetermined pipetting sequence and the predetermined pipetting amounts. Such a test can be carried out only by trained personnel under laboratory conditions. The known procedure cannot be used at home by laypersons or in emergency situations under time pressure and without optimal technical equipment.

It is the task of the present invention to further refine a generic type of test apparatus so that more sensitive measurements can be carried out.

A further task of the present invention is to provide a method for producing such a test apparatus.

Finally, another task of the present invention is to provide a test method that is simple to use and at the same time is highly sensitive.

### SUMMARY OF THE INVENTION

The first-mentioned task is solved in conjunction with a test apparatus for detecting an analyte (A) contained in a sample liquid, comprising a dry porous carrier (10) on which a selective binder (12) is arranged in a reaction zone, said binder being capable of selectively binding the analyte (A) once the carrier (10) has been wetted with the sample liquid, characterized in that, in the reaction zone (20), the following are further arranged: a complex made up of a biomarker and the first, complexed reaction partner of a pair of reporters consisting of a reporter enzyme and a reporter substrate that interact to generate an optically detectable signal, as well as the second, free reporter partner of the pair of reporters, in which the biomarker is equivalent to the analyte with regard to the selectivity of the binding capability of the selective binder, and in which binding of the selective binder with the complexed biomarker impedes the interaction between the complexed and the free reporter partner.

The second-mentioned task is solved in method for producing a test apparatus for detecting an analyte (A) contained in a sample fluid, comprising the steps of providing the following reaction elements: a selective binder (12) capable of selectively binding the analyte (A); a complex made up of a biomarker (B) and the first, complexed reporter partner of a reporter pair consisting of reporter enzyme (E) and reporter substrate (S) that interact to generate an optically detectable signal, the second, free reporter partner of the reporter pair, wherein the biomarker (B) is equivalent to the analyte (A) and is competitive with the latter regarding the selectivity of the binding capability of the selective binder (12), and wherein a binding of the selective binder (12) with the complexed biomarker (B) impedes the interaction between the complexed and the free reporter partner, characterized by the step of applying all the reaction elements in a common reaction zone on a porous carrier, said reaction elements, in the dry state of the carrier, being fixed thereon and at least two of the reaction elements being freely mobile in the porous carrier in its wetted state after the liquid sample has been applied.

The third-mentioned task is solved by a test method for detecting an analyte (A) in a liquid sample, comprising the steps of: providing a test apparatus as defined above; wetting of the reaction zone (20) with the liquid sample; and after the passage of a predetermined reaction time, detecting the presence or absence of an optical signal generated in the reaction zone (20) by the conversion reaction of the reporter partners.

### BRIEF DESCRIPTION OF THE DRAWINGS

Further features and advantages of the present invention may be obtained from the following specific description and the drawings which show:

FIG. 1: a diagrammatic view of a first biochemical variant of the test apparatus according to the invention.

FIG. 2: a diagrammatic view of a second biochemical variant of the test apparatus according to the invention.

FIG. 3: a diagrammatic view of a third biochemical variant of the test apparatus according to the invention.

FIG. 4: a diagrammatic view of a fourth biochemical variant of the test apparatus according to the invention.

FIG. 5: a first variant of a production method according to the invention.

FIG. 6: a second variant of a production method according to the invention.

FIG. 7: a third variant of a production method according to the invention.

FIG. 8: a diagrammatic sectional view of a first embodiment of an apparatus according to the invention.

FIG. 9: a diagrammatic sectional view of a second embodiment of an apparatus according to the invention.

FIG. 10: a diagrammatic sectional view of a third embodiment of an apparatus according to the invention.

### DETAILED DESCRIPTION OF THE INVENTION

The basic concept of the present invention is to combine a highly sensitive test method that is comparable to the known method of detection with the handling advantages of the lateral flow test. In addition, the present invention constitutes a simplification compared with the handling and production of the lateral flow test in that all the reaction elements are arranged in a single reaction zone. On the one hand, the waiting time for the user is avoided and on the other hand there is also no longer any uncertainty as to whether an apparently negative test result might be due to an interruption of capillary flows. In the case of the known lateral flow test, a separate control zone is proposed for this purpose, thus making it considerably more complicated and expensive to produce the test. In addition, handling is significantly simplified so that the error potential is greatly reduced, especially with regard to false-negative results that occur in the known lateral flow test, e.g. when the detection zone is accidentally wetted with sample liquid.

As mentioned, the biomarker can be the analyte itself. This guarantees the maximum possible equivalence with respect to the binding of the selective binder with the analyte present in the sample liquid, however, synthesis of the complex can be difficult and expensive. It is therefore frequently more advantageous to use a fragment of the analyte rather than the analyte as a biomarker. The fragment in question is preferably the one with which the selective binder interacts when it binds with the analyte. As another alternative, substances can be used that possess sections corresponding or similar to the binding site of the analyte for the selective binder. It is important that the complexed biomarker and the free analyte are substantially indistinguishable for the selective binder so that genuine competivity exists between the complexed biomarker and the analyte.

In keeping with the plurality of possible analytes, there is also a wide range of possibilities for designing the biomarker. It can, in particular, be a nucleic acid, a protein, a peptide, a low-molecular organic compound, a carbohydrate or a combination thereof.

Coupling the biomarker to a complex may alternatively be done with the reporter enzyme or the reporter substrate. It has proved especially advantageous to couple the biomarker with the reporter enzyme to the complex, because in this case, not only steric but also allosteric effects can be used to impede the enzymatic conversion of the substrate. Alkaline phosphatase, peroxidase, glucose-oxidase, β-galactosidase, malate dehydrogenase, glucose-6-phosphate dehydrogenase or lysozyme are examples of suitable reporter enzymes. Depending on the actual choice of reporter enzyme, a person skilled in the art must choose as the reporter substrate the substrate that is “suitable” for the respective enzyme.

In a special variant of the test according to the invention, the reporter enzyme is made up of at least two sub-units that are not enzymatically active individually and that are present separately on the dry, porous carrier. In this case, at least one sub-unit is not complexed with the biomarker. In this embodiment, binding of the selective binder with the complexed biomarker indirectly impedes the enzymatic conversion reaction, because binding of the enzyme sub-units to an active reporter enzyme is impeded. As will be explained in detail further below, this embodiment is especially advantageous with regard to achieving simple production of the test according to the invention.

Advantageously, the coupling of the complexed reaction components with each other, i.e. of the biomarker with the complexed reporter partner, is brought about by covalent binding. On the one hand, covalent binding offers the advantage of great stability, especially while the complex is in contact with the sample liquid; on the other hand, covalent binding guarantees close juxtaposition of the complexed components, and this particularly favours steric inhibition of the enzymatic reaction by the selective binder. Alternatively, however, other complex-forming mechanisms may also be used.

In particular a monoclonal or polyclonal antibody, a proteinogenic binder or a binder based on nucleic acids, particularly a so-called aptamer, are suitable as selective binders. The size of the selective binder is not limited to the structure necessary for the selective binding to be achieved with the biomarker or the analyte; instead, the selective binder can be a large-volume molecule or may be coupled with a large-volume molecule that interacts with the biomarker/reporter partner complex in a way that hinders the enzymatic reaction.

Advantageously, the selective binder is present on the dry, porous carrier in a form in which it is bound with the biomarker. As will be explained further below, this is particularly advantageous for the production of the test apparatus according to the invention. From a biochemical standpoint this can, however, be disadvantageous because the establishment of an equilibrium between the analyte and the complexed biomarker, with respect to the binding with selective binders, may be delayed and this would disadvantageously lengthen the duration of the test. In this respect, it may therefore be more advantageous if the selective binder is present separately from the complexed biomarker on the dry, porous carrier. In this variant, the biomarker and the analyte begin their competition for binding sites of the selective binder from the same starting position. An equilibrium can thus be more quickly achieved. This is particularly the case when a stable or even irreversible bond is established between the selective binder and the analyte or the biomarker.

In a particularly preferred embodiment of the present invention, provision is made for a reaction element from the group consisting of selective binder, complex and second reporter partner to be immobilized on the porous carrier or on an intermediate carrier positioned on the porous carrier, so that it is spatially fixed in the reaction zone also in the wetted state of the porous carrier. This variant of the test apparatus according to the invention offers two advantages. On the one hand, “bleeding” of the components from the reaction zone is avoided. On the other hand, it has been found that when the selective binder is the immobilized reaction element, the enzymatic conversion of the reporter substrate is significantly more efficiently impeded than when all the reaction elements are freely mobile in the porous carrier. It is assumed that the reason for this is the restriction of the spatial degrees of freedom caused by the immobilization, as a result of which the possible ways for the reporter partners to avoid, for example, steric inhibition, are limited. It has been found that immobilizing in particular the selective binder on an intermediate carrier, e.g. a glass, latex or plastic bead, produces a similar effect to that achieved by immobilization on the surface of the porous carrier itself. The use of an intermediate carrier may be favourable for the production method because the production of antibodies on beads, for example, is generally known and the mechanical fixing of beads on the porous carrier does not present any technical difficulties.

The immobilized reaction element is preferably fixed by covalent binding on the porous carrier or on the intermediate carrier. This results in particularly stable immobilization. The covalent bond may in this case be formed between the reaction element and a reactive group on the surface of the porous carrier or of the intermediate carrier. The following are particularly suitable reactive groups: an acid ester, an acid anhydride, an acid halogenide, an imide, an imidoyl ester, a carboxyl, a halogen carbonamide, a sulfonyl halogenide, an isothiocyanate, a thiol, a pyrimidyl sulfide, a halogen acetyl, a hydroxyl, a halogen alkyl, a phosphoramidite, an amine, a hydrazide, an azide, an aryl diazo, a nitrene, an aldehyde and/or a ketone.

One or more linking compounds may be interposed between the immobilized reaction element and the reactive group. Among others, a polyoxyalkyl unit, an aliphatic, a cycloaliphatic and/or an aromatic unit, in each case substituted or non-substituted, are particularly suitable as linking compounds.

As an alternative to the covalent binding, provision can be made for the immobilized reaction element to be fixed on the porous carrier, or on an intermediate carrier positioned on the porous carrier, by means of a non-covalent interaction between pairs of bridging substances. The following are examples of suitable bridging substances: complementary nucleic acid strands, streptavidin/biotin, avidin/biotin, streptavidin/Strep-tag, MBP/maltose, proteinA-IgG/antibody, hexa-His-tag/NTA, hexa-His-tag/anti-His-antibody, digoxigenin/anti-digoxogenin-antibody and/or GST/glutathione.

Provision may also be made in this variant for one or more linking compounds to be used that are preferably interposed between the porous carrier or the intermediate carrier and the partner of the pair of bridging substances that is coupled with it. Suitable linking compounds include, among others, those already mentioned above in connection with the covalent binding of the immobilized reaction element.

The porous carrier preferably consists of plastic, such as polystyrene or polyester, paper or another cellulose derivative, glass, metal, silicon, ceramic or a composite material thereof.

The porous carrier is preferably of planar construction, but it may also be designed as a three-dimensional, in particular a spherical carrier. In both variants the carrier can be mounted, in an advantageous embodiment, on a rod-shaped holding apparatus. With such an apparatus, it is possible to dip the carrier itself in the sample liquid in order to apply the sample liquid in the reaction zone.

As will be described in more detail further below in connection with the production method according to the invention, there may be a risk that some of the reaction elements react prematurely with one another. In an advantageous further refinement of the invention, provision is therefore made that at least one reaction element from the group comprising selective binder, complex and second reporter partner, is present in encapsulated form on the porous carrier. The encapsulated reaction element can, for example, be enclosed in capsules made of dextran or gelatine, and/or in liposome vesicles. Capsules made from dextran or gelatine are particularly suitable for arrangements of the inventive test method when it is used to examine aqueous sample liquids. This is because such capsules dissolve when in contact with the aqueous sample liquid and release the encapsulated reaction element. A similar situation exists when liposome vesicles are used for the encapsulation and the sample liquid is based on organic solvents.

In particular, the selective binder and/or the complex may be considered as the reaction element to be encapsulated. In such a case, premature binding between a selective binder and a complexed biomarker is reliably prevented.

In an advantageous embodiment of the invention, the common reaction zone is subdivided into a plurality of adjacent sub-zones, each of which carries one or more reaction elements of the group made up of selective binder, complex and second reporter partner. A single sub-zone can be designed as a gelatine layer enclosing the assigned reaction element or several assigned reaction elements. The gelatine layers may be arranged one on top of the other or alongside each other in the common reaction zone. The idea on which this embodiment is based is again that it will prevent a premature reaction of several reaction elements with one another. In this case, it is regarded as particularly advantageous to arrange the selective binder and the complex in different sub-zones. As an alternative to using a water-soluble layer of gelatine, sub-zones may also be designed as liquid-permeable films that enclose the assigned reaction element or the assigned reaction elements. Such films do not dissolve when they come into contact with the sample liquid; however, they do allow the reaction elements that they contain to be flushed out so that these elements can react jointly. Alternatively, the sub-zones may also be designed as paper layers that are impregnated with the assigned reaction element(s).

In order to produce a test apparatus according to the invention, provision, as is known from the prior art detection method mentioned at the beginning, is made first of all to make all the reaction elements available, i.e. the selective binder, the complex consisting of the biomarker and the first reporter partner, as well as the second reporter partner. However, instead of presenting these reaction elements in a solution and pipetting them as required in predetermined amounts and in a predetermined sequence, provision is made according to the invention to apply these elements on a dry, porous carrier in a common reaction zone so that they are fixed on the carrier in its dry state, and at least two of the reaction elements are freely mobile in the porous carrier after it has been wetted by applying the liquid sample. The application of reaction elements on a porous carrier so that they are either durably immobilized there, or are merely fixed in the dry state of the carrier and are freely mobile in the wetted state of the carrier, can be technically achieved in various ways, some of which are known to a person skilled in the art, some of which, however, are new. Nevertheless, up until now a corresponding application has not been carried out because of the prejudices held by experts in the field that a premature reaction of individual components with one another would be unavoidable. In particular, premature conversion of the reporter substrate by the reporter enzyme must be prevented. Also, in some applications it is necessary to keep the selective binder and the complexed biomarker separate from each other in order, when the test is used, to create a situation of equal competition between the analyte and the complexed biomarker, which can be advantageous with regard to the duration of the test.

In an advantageous variant of the production method according to the invention, provision is made for the application step to comprise several sub-steps of wetting the porous carrier with solutions that in each case contain one or more reaction elements dissolved in solvents, as well as at least one subsequent drying sub-step. Provision is made that in a temporally earlier sub-step a more strongly polar solution containing the reporter enzyme is used, and in a temporally later sub-step a more weakly polar solution containing the reporter substrate is used. The wetting of the porous carrier can be carried out for example by saturation, or by spraying or pressing the solution on. The wetting steps thus take place successively and with decreasing polarity. This means that initially the enzyme, which is active and easily soluble in an aqueous environment, is applied to the porous carrier. The application is preferably immediately followed by a drying step so that the enzyme is coupled to the porous carrier, for example, by adhesion forces.

If, in a subsequent step, the reporter substrate is applied in weakly polar or non-polar solution, e.g. toluene, the reporter enzyme that is active only in aqueous solution cannot convert the reporter substrate. In a subsequent drying step, the reporter substrate is then also fixed on the porous carrier, for example by adhesion forces.

The selective binder can be treated in similar fashion, unless it is applied together with the complex or is already bound to it.

In the case of a particularly advantageous choice of reaction elements in which the enzymatic conversion of the reporter substrate is completely or almost completely blocked by the binding of the selective binder with the complex consisting of the biomarker and first reporter partner, a single-stage production method is also possible in which a solution containing all the reaction elements is applied in the reaction zone, e.g. by saturation or by being sprayed or pressed on.

The fixing of the reaction elements purely by drying will, as a rule, mean that the reaction elements fixed in this manner are freely mobile in the porous carrier in its wetted state. However, as has already been explained, it has proved advantageous to firmly immobilize particularly the selective binder on a matrix so that it also remains spatially fixed in the wetted state of the porous carrier. Therefore, in a special embodiment of the production method according to the invention, provision is made for the immobilization to take place through the formation of a covalent bond between the immobilized reaction element and the porous carrier or an intermediate carrier. This can take place directly or indirectly via reactive groups on the surface of the porous carrier as well as with or without the interposition of one or more linking compounds between the immobilized reaction element and the reactive group. Reference is made to the above explanation of the apparatus according to the invention with respect to the favourable choice of reactive groups or linking compounds.

As an alternative to immobilization by covalent binding, provision can be made for the immobilization to be achieved by a non-covalent interaction between pairs of bridging substances, where in each case one partner of a pair of bridging substances is coupled with the reaction element and the other partner is coupled with the porous carrier or intermediate carrier. Here, too, one or more linking compounds may be interposed, particularly between the porous carrier or the intermediate carrier and the partner of the pair of bridging substances coupled with the carrier. Reference is made to the explanation given above of the apparatus according to the invention with respect to the favourable choice of bridging substances or linking compounds.

As an alternative or in addition to the application carried out by wetting steps with solutions of declining polarity, in a further refinement of the production method according to the invention, provision can be made for at least one of the reaction elements to be encapsulated prior to the application step. For this purpose, the reaction element(s) to be encapsulated can be enclosed in capsules of dextran or gelatine and/or in liposome vesicles. In this way it is also possible to prevent a premature undesired reaction of individual reaction elements with one another. Depending on the choice of capsule material, a person skilled in the art will of course have to take care to ensure that, when applying the reaction elements to the porous carrier, the solvent used does not attack the encapsulation.

As an alternative or in addition to the encapsulation of individual reaction elements, provision can be made for the application step to comprise the application of a plurality of layers in the common reaction zone, with each layer containing one or more reaction elements. It is particularly favourable to enclose the reporter substrate and the reporter enzyme in different layers in order to prevent premature enzymatic conversion and thus signal generation already at the production stage. It may also be desirable to apply the selective binder in its own layer, in order to create an equal starting situation for the competition between the analyte and the complexed biomarker for bonds with the selective binder. The layers can be arranged on top of or alongside each other in the reaction zone.

For example, the layers may be applied as gelatine layers that in each case enclose the assigned reaction element(s). This variant is especially favourable because it allows work to be performed on an aqueous basis throughout the entire manufacturing process.

Alternatively, the layers can also be applied in the form of liquid-permeable films. Such films do not necessarily dissolve on contact with the sample liquid, but they permit the intermixing of the reaction elements when wetted with the sample liquid.

Such films, but also layers of gelatine or other matrix materials, may be applied by cascade casting machines, which are known from the photographic industry for the production of colour films. Such machines create multi-layered structures in a one-stage operating step, and the individual layers can be applied in the form of highly viscous gels that contain a variety of fillers—in this case the different reaction elements—and no intermixing of the layers occurs because of their viscosity. The layered structure can optionally be subjected to a subsequent drying step.

Alternatively, different layers of paper, each of which is impregnated with one or more assigned reaction elements, may also be applied in the reaction zone. In this variant of the production method according to the invention, a larger number of operating steps is required; however, these are in each case particularly simple to carry out so that, overall, a very cost-effective and technically uncomplicated production method is achieved.

In addition to all the sub-zones having the same structure, it is also possible to have a combination of sub-zones of different types. A particularly advantageous embodiment of the method according to the invention, which results in a particularly advantageous embodiment of the apparatus according to the invention, is based on a paper layer that is impregnated with a first reaction element. The impregnation can be carried out, for example, by saturation with or spraying or pressing on a solution containing the first reaction element. Preferably after the paper layer has been dried, a layer containing a second reaction element, e.g. a gelatine layer or a liquid-permeable film, is applied on the upper side of the paper layer. Next, a further layer containing a third reaction element can be applied to the underside of the paper layer. In special cases, the third reaction element may also be identical to the second reaction element. Of course, it is also possible to coat the upper side and the underside of the paper layer simultaneously.

The present invention makes possible a new and particularly advantageous test method for detecting an analyte in a liquid sample. This test method comprises supplying a test apparatus according to the invention, wetting the reaction zone with the liquid sample and, after a given reaction time, detecting the presence or absence of an optical signal produced in the reaction zone by the conversion reaction of the reporter partners. The method may be used both quantitatively as well as non-quantitatively, with the quantitative determination of an analyte concentration in the sample being carried out by comparing a measured optical signal with suitable calibration values.

The technical implementation of the signal detection depends on the nature of the signal generated. Many enzyme/substrate pairs that are used to detect reactions lead to a coloration in the sense of increased absorption for light in a certain wavelength range. As an alternative, the enzymatic conversion of the substrate can also lead to the generation, amplification or weakening of a fluorescence or chemiluminescence signal. Depending on the type of optical signal, suitable means and methods of detection are known to a person skilled in the art.

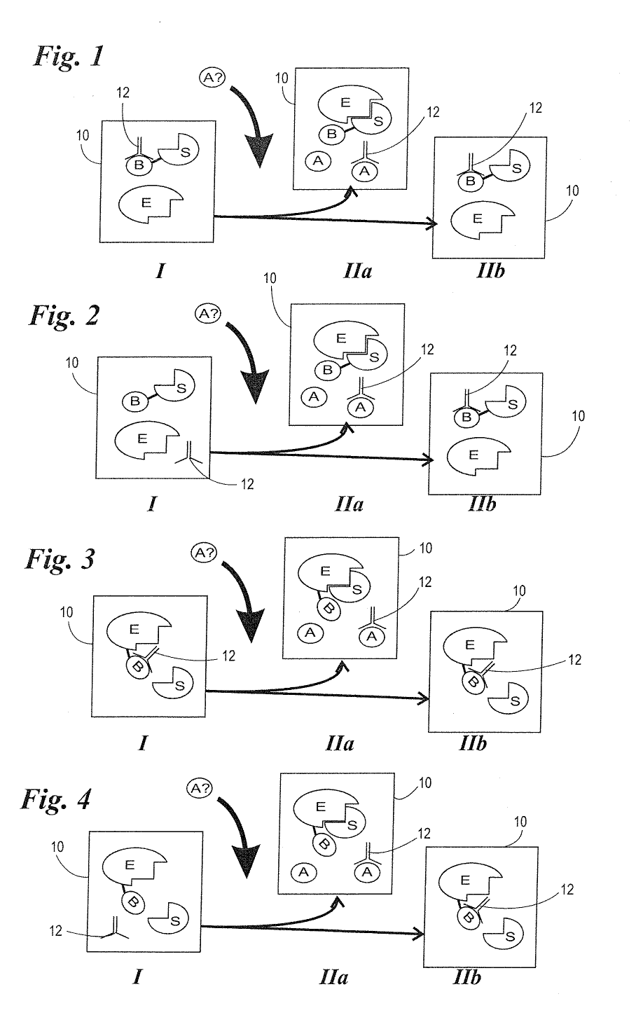

FIGS. 1-4 depict different variants of a biochemical test method that can be carried out with the test apparatus according to the invention. The structure of the presentation is the same in all four Figs. Sub-image I in each case shows a starting position of reaction components that are fixed on a dry, porous carrier 10. In each case, sub-image IIa shows the situation after adding a sample fluid containing an analyte. In each case, sub-image IIb shows the situation after adding a sample liquid that contains no analyte. The reaction components are a reporter enzyme E that can enzymatically convert a reporter substrate S, while generating an optically detectable signal. A further reaction component is a selective binder 12 that can be designed, for example, as a polyclonal or monoclonal antibody, in particular as a Fab, Fab2 fragment or as another recombinant antibody, such as, for example, a “single chain” antibody scFv. The use of other selective binders such as an aptamer is also possible. The selective binder 12 may also be derivatized with a voluminous molecule that is inert towards the enzymatic action of the enzyme E, but is helpful in causing the desired hindering of the enzymatic conversion of the substrate S. The voluminous molecule can, for example, be a synthetic polymer, a dendrimer, a natural substance, a nucleic acid, a peptide nucleic acid (PNA), a peptide, a protein, a lipid or a carbohydrate.

Alternatively, or in addition, the reporter substrate may also be coupled with such a voluminous molecule.

Finally, as a further reaction component, a biomarker B is provided that is equivalent to the analyte A with regard to its binding capability with the selective binder 12. In particular, the biomarker B itself may correspond to the analyte A.

FIGS. 1 to 4 in each case depict a typical test sequence in which a sample liquid, which presumably contains the analyte A, is applied in the reaction zone of the porous carrier 10 shown in sub-image I. This is indicated in the drawings by “A?”.

FIG. 1 shows an embodiment in which the biomarker B is complexed with the substrate S. This means that a stable and close bond is established between the biomarker B and the substrate S. A further special feature of the embodiment in FIG. 1 is that in the dry state of the porous carrier the selective binder 12 is already bound with the complexed biomarker B.

When analyte A is present in the applied sample liquid, analyte molecules compete with complex molecules for binding sites of the selective binder 12. It is necessary in this case for the initial binding of the binder with the biomarker B to be reversible. When there is an excess of analyte A, the binder 12 binds large amounts of analyte A while it releases the complexed biomarker B. As a result of the input of liquid, the enzyme E and/or the complex consisting of biomarker B and substrate S are suspended, so that at least one of these reaction elements is freely mobile in the porous carrier. This brings about an enzymatic conversion of the substrate S and, as a result, an optically detectable signal is generated.

The case in which no analyte is present in the sample liquid is depicted in sub-image IIb in FIG. 1. Here, the enzyme E and/or the complex are suspended by the input of liquid so that at least one of these elements is freely mobile in the porous carrier. Nevertheless, no enzymatic conversion of the substrate S takes place because an interaction between the enzyme E and the complexed substrate S is impeded by the binder 12 bound to the biomarker. In the diagramatically depicted case, this impeding involves complete suppression of the enzymatic effect due to steric inhibition. In other embodiments, allosteric effects may also be used. The above-mentioned derivatization of the binder 12 with a voluminous molecule is particularly helpful in the case of steric inhibition.

FIG. 2 shows a similar starting situation to that in FIG. 1, with the difference, however, that in the dry state of the porous carrier, the binder 12 is present separately from the biomarker B. This has the advantage that the competitive reaction of analyte A and complexed biomarker B for the binding sites of the selective binder 12 commences from a uniform starting situation. This can lead to an accelerated establishment of the equilibrium and thus to a shortening of the test duration. In addition, this variant is also suitable when the bond between the binder 12 and the analyte or the biomarker cannot be reversed, or only to a limited extent.

FIGS. 3 and 4 depict another basic variant of the present invention in which the biomarker B is not complexed with the substrate S but with the enzyme E. In FIG. 2, the binder 12 is already coupled with this complex in the dry state of the porous carrier, while in the variant shown in FIG. 4, it is separately present in the dry state of the porous carrier. Otherwise, the reaction takes place similarly to the reactions explained further above in connection with FIGS. 1 and 2.

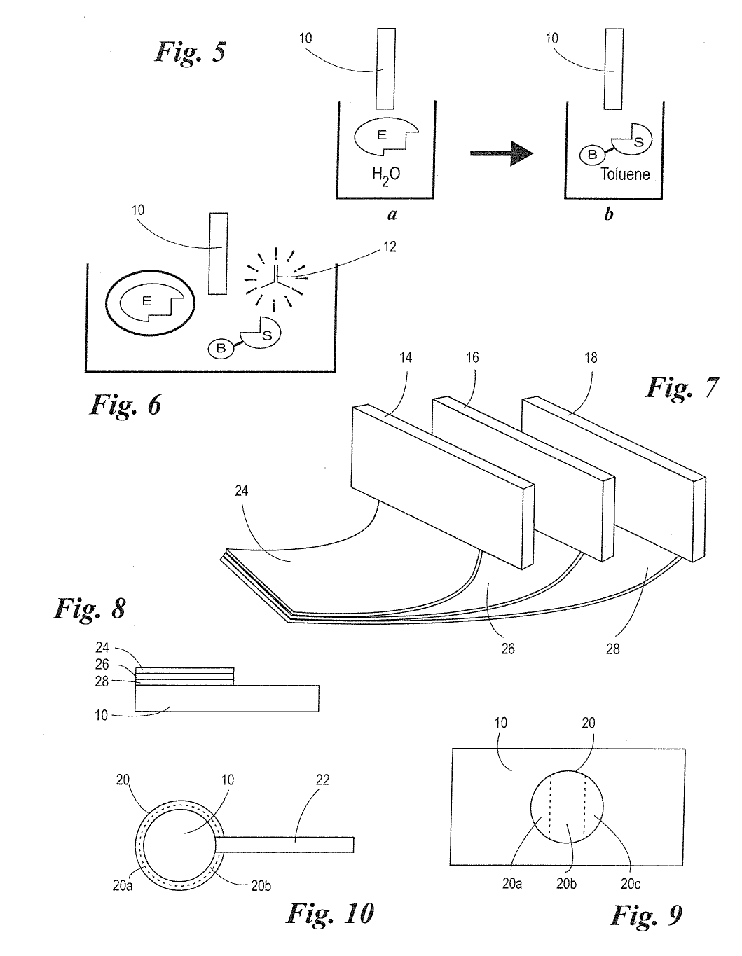

FIG. 5 shows a first embodiment of a method according to the invention for producing a test apparatus according to the invention. For this purpose, in a first step, a porous carrier 10 is saturated in an aqueous solution in which the enzyme E is present. Instead of using saturation, the solution can also be sprayed or pressed on. In a subsequent drying step, not separately shown, the enzyme E is fixed on the surface of the porous carrier by means of adhesion forces. This can be supported by suitable surface coating of the porous carrier. In a subsequent step, which is shown in FIG. 5b, the porous carrier 10 is again saturated using a toluene solution of a substrate S complexed with the biomarker B. The enzyme is not active in the non-polar toluene solution, so that, in this second procedural step, no enzymatic conversion of the substrate can occur. A subsequent drying step fixes the complex on the porous carrier. In a further step, not shown here, the selective binder also needs to be fixed on the porous carrier. This can take place before, between or after the steps depicted in FIG. 5, and preferably one further graduation occurs in the polarity of the solvent used so that premature binding of the binder to the biomarker B is prevented.

FIG. 6 shows a further embodiment of the production method according to the invention. Here the porous carrier 10 is saturated in a liquid that contains all the reaction elements, i.e. in the case depicted, the enzyme E, the binder 12 and a complex consisting of the substrate S and the biomarker B. In order to prevent a premature reaction of the individual elements among themselves, the enzyme E and the binder 12 are encapsulated in the embodiment shown. As examples of such encapsulations are shown, in the case of the binder 12, encapsulation in liposome vesicles, and, in the case of enzyme E, encapsulation in a gelatine or dextran capsule. In practical use, identical encapsulations are preferably used for both encapsulated elements. Otherwise, the application should take place in several steps, each one using suitable solvents.

FIG. 7 shows another embodiment of the production method according to the invention. In this case, using a cascade casting machine, of which only extruder elements 14, 16, 18 are shown, several highly viscous layers 24, 26, 28 are applied, each of which encloses one of the reaction elements (selective binder, complex consisting of biomarker and first reporter partner, second reporter partner). Because of the high viscosity, an exchange between the layers and thus a premature reaction of the reaction elements is excluded. The layers 24, 26, 28 can be designed in such a way that they dissolve when the sample liquid is introduced; alternatively, they may also be designed as liquid-permeable films. FIG. 8 shows a diagrammatic cross section through a test apparatus according to the invention, of the kind that is produced, for example, by a production method according to FIG. 7. Several layers 24, 26, 28, each of which containing a reaction element, are applied one on top of the other on a porous carrier 10. FIG. 9 shows a further embodiment of a test apparatus according to the invention, in which a reaction zone 20 is subdivided on a porous carrier 10 into several sub-zones 20 a, 20 b and 20 c, each of which containing a reaction element. In the embodiment in FIG. 9, the borders between the sub-zones are indicated by broken lines in order to indicate that such a subdivision of the reaction zone is not imperative. When the individual reaction elements are suitably applied, for example, in encapsulated form, the reaction zone 20 may also be uniformly conFig.d.

Finally, FIG. 10 shows a further embodiment of a test apparatus according to the invention, in which the porous carrier is designed as a three-dimensional spherical structure connected to a rod-shaped holding apparatus 22. The reaction zone (20) comprises the entire surface of the spherical carrier 10, and the broken line again indicates that subdivision into sub-zones (here two sub-zones 20a and 20b) is optional.

Of course, the embodiments shown in the specific description and in the Figs. are only illustrative examples of the present invention. In particular, the spatial design of the porous carrier, the choice of materials for the carrier, and the choice and design of the reagents, give a person skilled in the art a wide range of possibilities. In particular, the biochemistry used in each case must be adapted to the concrete requirements for detecting a specific analyte.

Now that the invention has been described, I claim:

1. A test apparatus for detecting an analyte (A) contained in a sample liquid, comprising a dry porous carrier (10) on which a selective binder (12) is arranged in a reaction zone, said binder being capable of selectively binding the analyte (A) once the carrier (10) has been wetted with the sample liquid, characterized in that, in the reaction zone (20), the following are further arranged:
a complex made up of a biomarker (B) and the first, complexed reporter partner of a reporter pair consisting of a reporter enzyme (E) and a reporter substrate (S) that interact to generate an optically detectable signal,
as well as the second, free reporter partner of the reporter pair
wherein the biomarker (B) is equivalent to the analyte (A) and competitive with the latter regarding the selectivity of the binding capability of the selective binder (12),
and wherein a binding of the selective binder (12) with the complexed biomarker (B) impedes the interaction between the complexed and the free reporter partner.

2. An apparatus according to claim 1, wherein the complexed reporter partner is the reporter enzyme (E), which reporter enzyme (E) is made up of at least two sub-units that are not enzymatically active individually and that are present separately on the dry, porous carrier (10), at least one of which not being complexed with the biomarker (B), and that a binding of the selective binder (12) with the complexed biomarker (B) impedes a binding of the sub-units to an active reporter enzyme (E).

3. An apparatus according to claim 1, wherein a reaction element of the group made up of the selective binder (12), the complex and the second reporter partner is immobilized by a covalent bond on the porous carrier (10) or on an intermediate carrier positioned on the porous carrier (10), said reaction element being spatially fixed in the reaction zone (20) also in the wetted state of the porous carrier (10).

4. An apparatus according to claim 3, wherein the covalent bond between the reaction element and a reactive group is formed on the surface of the porous carrier (10) or of the intermediate carrier.

5. An apparatus according to claim 3, wherein the covalent bond between the reaction element and a reactive group is formed on the surface of the porous carrier (10) or of the intermediate carrier, wherein one or more linking compounds are interposed between the immobilized reaction element and the reactive group.

6. An apparatus according to claim 1, wherein at least one reaction element from the group consisting of selective binder (12), complex and second reporter partner is present encapsulated, namely enclosed in capsules consisting of dextran or gelatine and/or in liposome vesicles, on the porous carrier (10).

7. An apparatus according to claim 1, wherein the common reaction zone (20) is subdivided into a plurality of adjacent sub-zones (24, 26, 28; 20a, 20b, 20c), each of which subzones carrying one or more reaction elements from the group consisting of selective binder (12), complex and second reporter partner, wherein at least one sub-zone (24, 26, 28) is designed as a gelatine layer enclosing the assigned reaction element(s).

8. A method for producing a test apparatus for detecting an analyte (A) contained in a sample fluid, comprising the steps of providing the following reaction elements:
a selective binder (12) capable of selectively binding the analyte (A)
a complex made up of a biomarker (B) and the first, complexed reporter partner of a reporter pair consisting of reporter enzyme (E) and reporter substrate (S) that interact to generate an optically detectable signal,
the second, free reporter partner of the reporter pair,
wherein the biomarker (B) is equivalent to the analyte (A) and is competitive with the latter regarding the selectivity of the binding capability of the selective binder (12), and wherein a binding of the selective binder (12) with the complexed biomarker (B) impedes the interaction between the complexed and the free reporter partner, and
wherein the step of applying all the reaction elements in a common reaction zone (20) on a porous carrier (10), said reaction elements, in the dry state of the carrier (10), being fixed thereon and at least two of the reaction elements being freely mobile in the porous carrier (10) in its wetted state after the liquid sample has been applied.

9. A method according to claim 8, wherein the step of applying comprises several sub-steps of wetting the porous carrier (10) with solutions, each of which contains one or more reaction elements dissolved in solvent, as well as at least one subsequent drying sub-step, with a more strongly polar solution containing the reporter enzyme (E) being used in a temporally earlier sub-step, and a more weakly polar solution containing the reporter substrate (S) being used in a temporally later sub-step.

10. A method according to claim 8, wherein a reaction element is immobilized on the porous carrier (10) or on an intermediate carrier positioned on the porous carrier (10) in such a way that it also remains spatially fixed in the wetted state of the porous carrier (10), wherein the immobilizing is achieved by forming a covalent bond between the immobilized reaction element and the porous carrier (10) or the intermediate carrier.

11. A method according to claim 10, wherein the covalent bond is formed between the reaction element and a reactive group on the surface of the porous carrier (10) or of the intermediate carrier.

12. A method according to claim 10, wherein the covalent bond is formed between the reaction element and a reactive group on the surface of the porous carrier (10) or of the intermediate carrier, wherein one or more linking compounds are interposed between the immobilized reaction element and the reactive group.

13. A method according to claim 8, wherein at least one of the reaction elements is encapsulated, namely enclosed in capsules made of dextran or gelatine and/or in liposome vesicles, before the application step.

14. A method according to claim 8, wherein the application step comprises the application of a plurality of layers in the common reaction zone (20), each layer containing one or more reaction elements, wherein at least one of the layers is applied as a gelatine layer enclosing the assigned reaction element(s).

15. A test method for detecting an analyte (A) in a liquid sample, comprising the steps of:
providing a test apparatus according to claim 1,
wetting of the reaction zone (20) with the liquid sample,
after the passage of a predetermined reaction time, detecting the presence or absence of an optical signal generated in the reaction zone (20) by the conversion reaction of the reporter partners.
